(19) United States
(12) Patent Application Publication
(10) Pub. No.: US 2006/0261285 A1
(43) Pub. Date: Nov. 23, 2006

(54) Toothbrush sterilizer

(76) Inventor: Richard F. Broerman, Evansville, IN (US)

Correspondence Address: Richard F. Broerman, 512 Wyndclyff, Evansville, IN, 47711, US

(21) Appl. No.: 11/097,885

(22) Filed: Apr. 4, 2005

(51) Int. Cl. A61L 2/10

(52) U.S. Cl. 250/455.11

(57) ABSTRACT

The invention discloses a toothbrush sterilizer that sterilizes the toothbrush with ultraviolet light from a Light Emitting (LED) utilizing a battery and electronics.

### BACKGROUND OF THE INVENTION

1. Field of the Invention

The present invention relates to generally sterilizing of a toothbrush. This is done by the use of a Light emitting diode (LED). The diode emits ultraviolet light, which kills germs.

2. Description of Prior Art

There are other means of sterilizing toothbrushes. Typical of these is:
U.S. Pat. No. 6,461,568 issued to Eckhardt on Nov. 8, 2002
U.S. Pat. No. 6,171,559 issued to Sanders on Jan. 9, 2001
U.S. Pat. No. 6,096,264 issued to Peifer on Aug. 1, 2000
U.S. Pat. No. 5,487,877 issued to Choi on Jan. 9, 1996
U.S. Pat. No. 4,888,487 issued to Ritter on Dec. 19, 1989
U.S. Pat. No. 4,906,851 issued to Beasley on Mar. 6, 1990

Methods and apparatus for sterilizing small objects. A small object is inserted into a chamber formed in the sterilizer. A portion of the small object mates with a lockout device to disable the lockout device. After the lockout device is disabled, the sterilizer produces at least one flash of high intensity ultraviolet light produced by an ultraviolet light source. The ultraviolet light flash kills microorganisms on the surface of the small object. The chamber of the sterilizer may be lined with a reflective coating to disperse the ultraviolet light around the chamber so that most, if not all, of the small object within the sterilizer is exposed to the ultraviolet light.

A toothbrush sterilization unit for sterilizing the bristle-head of at least one toothbrush with a disinfectant. The toothbrush sterilization unit includes a housing having an interior compartment. The interior compartment includes a plurality of docking stations for holding in place a plurality of toothbrushes. The housing includes a pumping system assembly for dispensing a disinfectant on the bristle-heads of the toothbrushes during a dispensing cycle. The housing also includes a rotary fan and a heating member for drying the disinfectant on the bristle-heads of the plurality of toothbrushes during a drying cycle.

A sterilizing cabinet for storing and sterilizing personal toilet articles, such as toothbrushes. The cabinet has a housing including a swinging door, receptacles for receiving and supporting a variety of differently configured electrically operated toothbrushes, and a rack having holes or slots for receiving and supporting manual toothbrushes. The cabinet has an electrical system having and supplying a sterilizing lamp capable of emitting ultraviolet radiation, an illuminating lamp capable of emitting visible light, and an AC-to-DC converter connected to recharging elements disposed within the receptacles for receiving electric toothbrushes. The sterilizing and illuminating lamps are independently switched. A safety switch senses door closure, and breaks power to the sterilizing lamp when the door is open. A charging receptacle connected to the AC-to-DC converter is mounted on the exterior of the cabinet.

The present invention relates to a restroom organizer and toothbrush sterilizing apparatus (1) which includes a toothbrush sterilizer (4), a toothpaste dispenser (2) a soap supplier (7a) and a control circuit (11). Various sanitizers (4, 31, 80, 100) are disclosed for disinfecting and deodorizing various articles and of the atmosphere. The invention also discloses various compartments (40, 50, 82, 84) for storing items to be sanitized.

A toothbrush conditioner includes a body having an upper end. A cover member is removably mounted to the upper end for therewith providing a conditioning chamber. A support plate is positioned within the body member for selectively positioning a toothbrush to be conditioned within the chamber. An ultraviolet radiation source is carried by the cover member and is movable therewith for being selectively positioned within the chamber proximate the brush to be conditioned when the cover member is mounted to the upper end. A plurality of vent openings are in the body member. A control device is in connection with the radiation source for causing selective intermittent operation thereof.

A device for sterilizing and storing toothbrushes is provided which includes a container for holding the toothbrushes therein whereby current to a ultraviolet ray lamp that supplies an application of germicidal radiation to bristles of toothbrushes stored therein can be automatically deenergized when the container is in an opened position at a predetermined distance.

While these toothbrush sterilizers may be suitable for the purpose for which they were designed, they would not be suitable for the purposes of the present invention, as hereinafter described.

### SUMMARY OF THE PRESENT INVENTION

A primary object of the present invention is to provide a means to sterilize toothbrushes.

Another object of the present invention is to provide a means to sterilize toothbrushes utilizing ultraviolet light.

Another object of the present invention is to provide a means to sterilize toothbrushes utilizing Ultraviolet Light Emitting Diodes (LED's).

Another object of the present invention is to provide a means to sterilize toothbrushes utilizing battery power.

Another object of the present invention is to provide a toothbrush sterilizer that is inexpensive to manufacture and operate.

Another object of the present invention is to provide a toothbrush sterilizer with electronic controls to cause the Ultraviolet Light Emitting Diodes (LED's) to turn on and off while sterilizing the toothbrush.

Another object of the present invention is to provide a toothbrush sterilizer with electronic controls to cause the Ultraviolet Light Emitting Diodes (LED's) to be off most of the time to conserve battery life.

The present invention overcomes high power consumption.

The present invention overcomes short ultraviolet light source life.

The present invention overcomes short battery life.

The foregoing and other object and advantages will appear from the description to follow. In the description reference is made to accompanying drawings, which forms a part hereof, and in which is shown by way of illustration specific embodiments in which the invention may be practiced. These embodiments will be described in sufficient detail to enable those skilled in the art to practice the invention, and it is to be understood that other embodiments may be utilized and that structural changes may be made without departing from the scope of the invention. In the accompanying drawing, like reference characters designate the same or similar parts throughout the several views.

### LIST OF REFERNCE NUMERALS UTILIZED IN THE DRAWINGS

10 Toothbrush
12 Case
14 Light Emitting Diode (LED) Ultraviolet
16 Battery
18 Electronics
20 Wiring

### BRIEF DESCRIPTION OF THE DRAWING FIGURES

In order that the invention may be more fully understood, it will now be described, by way of example to the accompanying drawing in which:

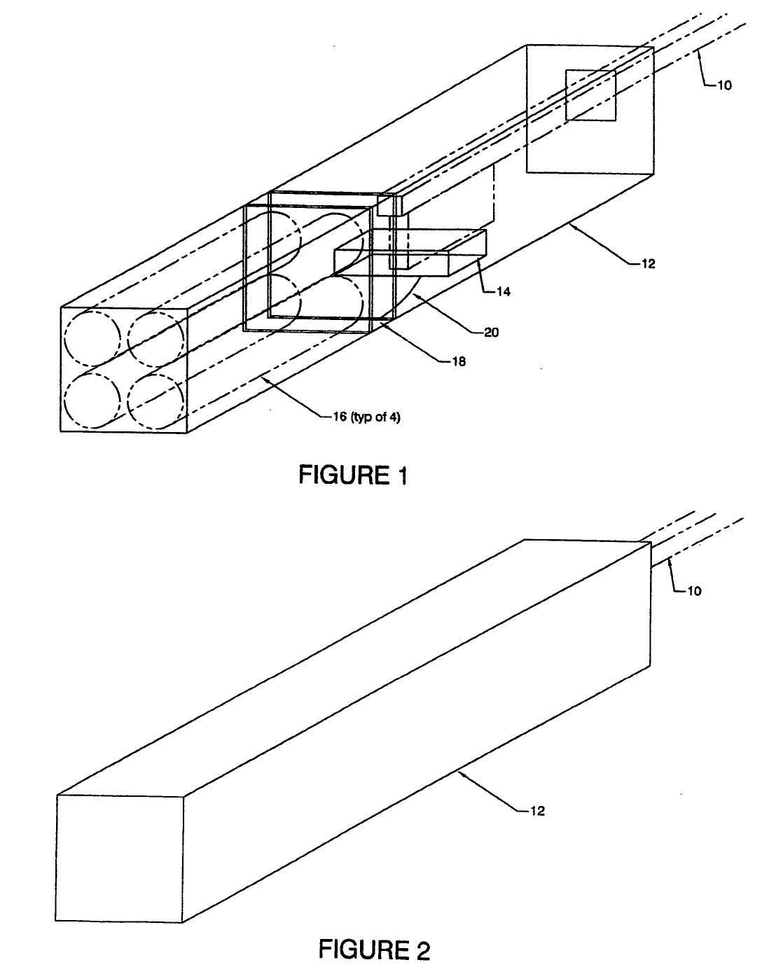

FIG. 1 is an illustrative view of the present invention with a toothbrush inserted and with the cover removed.

FIG. 2 is an illustrative view of the present invention with the cover in place.

### DETAILED DESCRIPTION OF THE PREFERRED EMBODIMENTS

The following discussion describes in detail one embodiment of the invention and several variations of that embodiment. This discussion should not be construed, however, as limiting the invention to those particular embodiments. Practitioners skilled in the art will recognize numerous other embodiments as well. For a definition of the complete scope of the invention, the reader is directed to the appended claims.

FIG. 1 is an illustrative view of the toothbrush sterilizer with the cover removed. The present invention consists of a case 12 to hold a toothbrush 10, a Light Emitting Diode (LED) that emits ultraviolet light 14, a battery 16, electronics 18, and wiring 20.

FIG. 2 is an illustrative view of the toothbrush sterilizer with the cover installed.

What is claimed is new and desired to be protected by Letters Patent is set forth in the appended claims:

1. A structure for sterilizing a toothbrush.

2. The device as recited in claim 1, wherein the device for an enclosing housing having a door for access to the inside of the housing

3. The device as recited in claim 2, wherein the device contains a light emitting diode led emitting ultraviolet light.

4. The device as recited in claim 3, wherein the device contains a battery.

5. The device as recited in claim 4, wherein the device contains electronics to switch the said “led” on and off at predetermined intervals.
